(19) United States
(12) Patent Application Publication
(10) Pub. No.: US 2024/0070990 A1
(43) Pub. Date: Feb. 29, 2024

(54) INJECTING EMOTIONAL MODIFIER CONTENT IN MIXED REALITY SCENARIOS TO MIMIC REAL LIFE CONDITIONS

(71) Applicant: INTERNATIONAL BUSINESS MACHINES CORPORATION, ARMONK, NY (US)

(72) Inventors: Robert Huntington Grant, Marietta, GA (US); Zachary A. Silverstein, Georgetown, TX (US); Jeremy R. Fox, Georgetown, TX (US); Todd Russell Whitman, Bethany, CT (US)

(21) Appl. No.: 17/821,554

(22) Filed: Aug. 23, 2022

(51) Int. Cl. G06T 19/00; A61B 5/16

(52) U.S. Cl. CPC G06T 19/006; A61B 5/165

(57) ABSTRACT

According to one embodiment, a method, computer system, and computer program product for mixed-reality emotional modification is provided. The present invention may include collecting biometric information on a user; identifying a mental state of the user based on the biometric information; and, responsive to determining that the mental state does not match an expected baseline associated with a mixed-reality experience, modifying the mixed-reality experience with one or more virtual content elements.

### BACKGROUND

The present invention relates, generally, to the field of computing, and more particularly to mixed reality.

Mixed reality is a field concerned with merging real and virtual worlds such that physical and digital objects co-exist and interact in real time. Mixed reality does not exclusively take place in either the physical or virtual worlds but is a hybrid of reality and virtual reality; as such, mixed reality describes everything in the reality-virtuality continuum except for the two extremes, namely purely physical environments, and purely virtual environments. Accordingly, mixed reality includes augmented virtuality (AV), augmented reality (AR), and virtual reality (VR). Mixed reality has found practical applications in such areas as remote working, construction, gaming, and military, academic and commercial training.

### SUMMARY

According to one embodiment, a method, computer system, and computer program product for mixed-reality emotional modification is provided. The present invention may include collecting biometric information on a user; identifying a mental state of the user based on the biometric information; and, responsive to determining that the mental state does not match an expected baseline associated with a mixed-reality experience, modifying the mixed-reality experience with one or more virtual content elements.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment;

FIG. 2 is an operational flowchart illustrating a mixed-reality emotional modifier process 200 according to at least one embodiment; and

FIG. 3 is an operational flowchart illustrating a profile creation process 300 according to at least one embodiment.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention relate to the field of computing, and more particularly to mixed reality. The following described exemplary embodiments provide a system, method, and program product to, among other things, create an emotional profile tailored to a user, and inject emotionally-influencing situation-specific content into a mixed reality environment of the user based on the profile to maintain the user's emotional state at an expected baseline.

As previously described, mixed reality is a field concerned with merging real and virtual worlds such that physical and digital objects co-exist and interact in real time. Mixed-reality systems use software to generate images, sounds, haptic feedback, and other sensations to augment a real-world environment. While the creation of this augmented environment can be achieved with mobile devices such as cell phones or tablets, more specialized equipment is also used, typically in the form of glasses or headsets where computer generated elements are overlaid onto a view of the real world by being projected or mapped onto a lens in front of a user's eyes. With the help of computer augmentation, information about the surrounding world of the user, as well as other digital elements overlaid onto the world, become interactive and digitally manipulable.

Augmented reality environments may be utilized to immerse a user in environments authored with the intention of invoking and/or maintaining particular emotional responses, such as a training simulation, a game, a narrative experience such as a movie, a social platform, et cetera. For example, a mixed-reality game may offer a mixed-reality experience to the user that is expected to keep the user's emotional state around a particular expected baseline. The expected baseline of a game may represent an intended user experience of the game and may be based on such factors as the desired atmosphere, emotional arc of the story, genre, type of gameplay, et cetera. For example, in a high-intensity horror game, the user may be expected to maintain a state of agitation. However, if the user's emotional state deviates from the expected baseline, the user may have a negative experience with the game. For example, if the user exceeds that state of agitation by too much, the user may be too frightened, or too stressed, and may experience degraded enjoyment of the game. Conversely, if the user falls below that expected baseline of agitation, the user may not be engaged with or challenged by the game, and may also have a negative experience with the game. Keeping a user's emotional state at the expected baseline may improve the user's engagement, enjoyment, and overall experience with the MR game.

In another example, when participating in an augmented-reality or virtual-reality training program designed to simulate a stressful situation such as a confrontation with an unruly individual or a natural disaster, a user may be expected to maintain a state of calm which represents how a user ought to be feeling in a real-world situation corresponding to the training simulation; if the user becomes agitated and exceeds that state of calm by too much, the user may fail to act or act incorrectly due to errors resulting from the user's heightened emotions. Conversely, if the user falls below that expected baseline of calm, the user may in fact be too calm; a certain amount of agitation may be expected from a normal human being in a stressful environment, such as the environment corresponding to the training simulation, and if the user's emotional state is too far below the expected emotional state, the user may not be engaged with the simulation, may be sleepy, et cetera, and may therefore fail to act, act incorrectly, or fail to appropriately engage with or learn from the content of the training simulation. Keeping a user's emotional state at the expected baseline may improve the user's decision-making abilities, engagement, happiness, and overall experience with the mixed reality training material, and may improve the efficacy of the training simulation with respect to the user.

In another example, a narrative experience such as a movie may involve a number of story beats; the author of the narrative experience may expect a certain default emotional state at each or a subset of story beats. For example, at the climax of the movie, an author may expect a user to feel a certain level of excitement, and at a death scene, the author may expect a user to feel a certain level of sadness. If the user's emotional state conforms to the expected emotional baselines, the user may more fully enjoy the narrative, whereas if the user deviates from the expected baseline the user may be overstimulated by or unengaged with the experience.

As such, it may be advantageous to, among other things, implement a system that can measure a user's emotional state during a mixed reality session, identify emotional content that influences a user's emotional state, and create a profile that maps the relationships between emotional content and the user's emotional state. The system may then measure the user's emotional state during a mixed reality session against an expected baseline associated with that mixed reality session, and modify the mixed reality session with the emotional content based on the profile if the user's emotional state deviates significantly from the expected baseline. Therefore, the present embodiment has the capacity to improve the technical field of mixed reality by maintaining a user's emotional state against an expected baseline and thereby improving a user's immersion in a mixed reality environment, and improving the efficacy of mixed reality training.

According to at least one embodiment, the invention is a method of creating a profile mapping relationships between virtual content elements and an emotional state of a user based on monitoring the user's emotional state during a mixed reality session.

According to at least one embodiment, the mixed reality session may be a discrete period of time where the user is interacting with a particular mixed reality program or experience through a mixed reality device. The mixed reality program may be a software program such as a training simulator, a game, a narrative experience such as a movie, a social platform, et cetera that creates or provides one or more mixed-reality experiences for the user to interact with through a mixed reality device. The mixed reality experiences may be discrete episodes comprising one or more scenes, virtual objects, narrative elements, graphical elements, simulated characters, et cetera; the mixed reality experiences may include training simulations, virtual tours, story vignettes, chapters of a game, et cetera. The mixed reality device may be any device or combination of devices enabled to record real-world information that the mixed reality program may overlay with computer-generated perceptual elements to create the mixed-reality reality environment; the mixed reality device may further record the actions, position, movements, et cetera of the user, to track the user's movement within and interactions with the mixed reality environment. The mixed reality device may display the mixed reality environment to the user. The mixed reality device may be equipped with or comprise a number of sensors such as a camera, microphone, accelerometer, et cetera, and/or may be equipped with or comprise a number of user interface devices such as displays, touchscreens, speakers, et cetera. In some embodiments, the mixed reality device may be a headset that is worn by the viewer.

According to at least one embodiment, the emotional state of a user may be a measure of one or a range of multiple emotions such as anger, calmness, sadness, anxiety, boredom, fright, et cetera experienced by a user at any given point during a mixed-reality session. The emotional state of a user may be assessed based on biometric data of the user. Each emotion assessed as part of the emotional state may further possess a magnitude, which may be a number indicating the strength of the emotion.

Biometric data of the user may be data pertaining to unique physical or behavioral characteristics of the user, such as pupil width, eye movement, stress responses, heart rate, breathing rate, et cetera. Biometric information, as referred to herein, may also encompass the position and movements of the user, as well as sounds and speech of the user relevant to determining mental state. Biometric information may be collected from the user only after obtaining a user's express permission. Biometric information may be collected by one or more sensors such as pupillometry sensors, infrared and visible light cameras, heart rate monitors, blood pressure monitors, et cetera, and may be standalone or may be integrated into devices such as wearable vitality trackers, mixed reality devices, mobile devices, et cetera. Biometric information of a user may be collected continuously or at regular intervals during a mixed reality session and/or between mixed reality sessions. In some embodiments of the invention, biometric information may be collected at points indicated by an author of a mixed-reality experience where a user's emotional state is intended to be at an expected baseline, such as the story beats of a narrative experience.

In some embodiments of the invention, the system may capture baseline biometric readings of a user in a control iteration. The control iteration may involve immersing a user in a control mixed-reality environment that is designed not to provoke any emotional response from the user by default and to monitor the user's biometric information while the user is within the environment, thereby assessing a default emotional state. In some embodiments of the invention, the system may administer virtual content elements generically associated with certain emotions to the user in the control mixed-reality environment while measuring biometric information of the user, thereby preliminarily assessing which virtual content elements provoke which changes in the emotional state of the user, and the strength of such changes. In some embodiments of the invention, the default emotional state may be pre-determined based on statistical data, and/or based on non-biometric user information such as interests, feedback, demographic information, et cetera, and/or may be pre-provided by a programmer, the user, or an author of a mixed reality program or environment.

The system may assess the emotional state of the user based on the biometric information by comparing the biometric information gathered during the mixed-reality session against the default emotional state. The system may perform this assessment upon initialization of the mixed-reality experience, in response to biometric information being received from sensors, at regular intervals, et cetera. Biometric information is not the user's emotional state, but elements of biometric information may individually or in combination indicate an emotional state in the user. For example, a high heart rate indicates stress; a high heart rate and a smile may indicate excitement/joy, and a high heart rate and shouting or violent motion may indicate anger. The system may initially use generic relationships between a generic user's biometric information and a generic user's mental state based on statistical analysis which may be pre-provided, extracted from crowdsourced data, crawled from a repository, et cetera, and may adjust the relationships over time based on the user's biometric information to better represent the connection between the biometric information and mental state of the individual user. The system may use a machine learning model trained on generic relationships to identify the mental state of the user based on the user's biometric information. This machine learning model may be set up as a master baseline knowledge corpus as the system is set up for use, prior to initialization of the system.

According to at least one embodiment, the profile may be a list of virtual content elements and the one or more emotions that each virtual content element evokes in a user, and potentially the magnitude to which the virtual content element evokes that emotion. The magnitude may be expressed as a number, which represents the amount to which the user's emotional state is affected by the virtual content in one or more contexts. The virtual content elements may be video, imagery, and/or audio elements that may be injected into a mixed reality session to influence the emotional state of the user. Virtual content elements may, for example, include uplifting text, music that makes the user feel happy or sad, colors or color schemes that make a user feel more agitated or more relaxed, cramped fonts or harsh dialogue that increase user anxiety, environmental effects such as rain that soothe a user, images that the user feels nostalgic for, et cetera. In some embodiments of the invention, virtual content elements may be characteristics of the mixed-reality environment or program itself, such as a difficulty level of a game, volume of audio, size, and/or behavior of a simulated crowd around the user, behavior of simulated characters, et cetera. The profile may further comprise metadata associated with the virtual content elements providing identifying information about each virtual content element, such as for example the type of virtual content element (audio, video, gameplay, et cetera), the emotion or emotions evoked by the virtual content element in generic users and/or the user, the mixed reality programs or experiences associated with the virtual content element and/or within which the virtual content element could be injected, et cetera.

In some embodiments, virtual content elements may initially be selected for the profile from a list and/or repository based on the effect that a virtual content element has on a generic user as derived through statistical analysis, or the effect that a virtual content element has on a generic user with one or more matching interests or traits of the user; the system may then, for example by introducing the content element to the user in the control mixed-reality environment or in other mixed-reality environments, assess the particular effect that the virtual content element produces on the mental state of the user. In some embodiments, the initial virtual content elements may be pre-provided to the system, for example by the user, by a human administrator or programmer, et cetera. According to at least one embodiment, the mapping of the virtual content elements to a generic user's mental state may be based on public historical responses which could be cataloged based on a larger population. Within certain scenarios, the use of crowdsourcing emotional responses may be utilized for certain types of content requirements. Based on the expected response from a demographic of generic users, the system may contextually utilize virtual content elements to evoke certain emotional responses from the user. In some embodiments of the invention, the system may utilize private interactions for statistical analysis of crowdsourced emotional responses, by scrubbing the data of any personal information pertinent to the individual from which the data originated and anonymizing the data for statistical purposes of tool usage and metrics. In some embodiments of the invention, the system may analyze social media platforms for feedback and comments which could help indicate situations that cause a certain desired emotional response. For example, the system may identify that a picture of a bug has inspired the remark “ewww” in the comments; the system may associate that photo of a bug with a disgusted/stressed emotional response, and may add pictures, videos, digital models et cetera of bugs into a mixed-reality scenario to evoke a more stressed response. In some embodiments of the invention, virtual content elements may be associated with particular mixed reality programs or experiences; the system may select virtual content elements associated with a given mixed reality program or experience based on determining that the user owns, has access to, and/or is initializing an instance of that mixed reality program or experience. The system may also select virtual content elements to add to the profile as they are added or become available to the system.

According to at least one embodiment, the invention is a method of measuring an emotional state of a user during or prior to a mixed reality session, matching the measured emotional state against an expected baseline associated with that mixed reality session, and responsive to determining that the measured emotional state has deviated from the expected baseline by a threshold amount, modifying the mixed reality session with virtual content based on a profile.

According to at least one embodiment, the expected baseline may be an emotional state that is associated with a given mixed-reality program and/or mixed reality environment. The expected baseline may be an emotional state that an author of the mixed-reality program or mixed-reality environment expects an emotional state of a user to conform to immediately prior to initializing, for the duration of, or at one or more points within, the mixed reality experience. In some embodiments of the invention, the expected baseline may reflect an emotional state at which the user would best enjoy the mixed reality content; in some embodiments, for example in mixed-reality training simulations, the expected baseline may reflect a starting emotional state that the user should be at prior to initializing the mixed reality experience; in the example of a training simulation for an emergency medical technician involving treating patients at the scene of an accident, the expected baseline for beginning the scenario may be a state of alertness or light anxiety, as a user entering the scenario in a tranquil or highly agitated emotional state may not be emotionally disposed to effectively carry out the training scenario. In some embodiments, the system may only allow a user to begin a mixed reality experience such as a training scenario if the user's emotional state falls within the threshold values or otherwise conforms to the expected baseline, and may disable a user's access to the mixed-reality experience.

In some embodiments of the invention, the expected baseline may be an aspirational emotional state that the mixed reality experience is designed to teach the user attain, such as in the case of a firefighting training simulation that is teaching firefighters to remain calm and collected in the presence of dangerous conflagrations. The expected baseline may be consistent throughout a mixed-reality experience; for example, in a brief, relaxing mixed reality experience of a nature walk, the expected baseline may be a state of consistent calm and relaxation throughout the experience. However, the expected baseline may also change; in a narrative-driven mixed-reality experience, the story may reach a climax by the end, and the expected baseline may change to reflect the heightening tension and excitement evoked by the rising action and climax of the narrative. In some embodiments of the invention, for example in a training simulation, the expected baseline may be dynamically assessed at intervals or based on the user's actions within the mixed-reality environment. For example, if the user detects a hazardous condition such as a fire or hostile simulated individual, the expected baseline may change to reflect the heightened tensions expected of the user. The author may be a creator or provider of mixed-reality content, or an individual associated with the mixed-reality content, such as a teacher teaching a course that comprises a mixed reality training simulation. In some embodiments of the invention, the expected baseline may be an emotional state comprising a plurality of different emotions at different magnitudes, or a plurality of different emotions that are expected to be present in a user.

According to at least one embodiment, for example, where the system calculates and monitors a magnitude of emotions comprising the emotional state, the system may determine that the measured emotional state has deviated from the expected baseline by a threshold amount. The threshold amount may represent a difference between the magnitude of an emotion of the emotional state of the user and a magnitude of that emotion within the expected baseline that is large enough to identify a deviation and conclude that the user is no longer conforming to the expected baseline and that additional action is necessary to restore the user's emotional state to the expected baseline. The threshold amount may be pre-provided, for example by the author, and/or may be determined by machine learning. The system may compare each emotion of the expected baseline against the corresponding emotions of the user's emotional state and may identify that the measured emotional state has deviated from the expected baseline if any one emotion exceeds the threshold. In some embodiments of the invention, the system may identify deviation if a plurality or a specified combination of emotions exceed the threshold. The system may compare the measured emotional state against the expected baseline at regular intervals, continuously/in real time/near real time, at the beginning/prior to commencement of a mixed reality session or experience, at points specified by the author, et cetera.

In some embodiments of the invention, for example where emotions comprising a mental state are identified and monitored but the magnitude of those emotions is not, the system may identify a deviation when one or more emotions comprising the expected baseline are not present in the emotional state. For example, if the user is not experiencing sadness during a scene where sadness is expected.

According to at least one embodiment, the system may, responsive to identifying a deviation, select one or more virtual content elements to add into the mixed reality environment. The system may identify which emotion or emotions deviated from the expected baseline, and the magnitude of the deviations, and assess what magnitude of effect must be produced on the user with respect to the deviating emotions to restore the emotions to the expected baseline. The system may consult the profile to determine which virtual content elements would produce the desired effect, based on their associated emotion, and/or the magnitude of effect they produce on the user or on generic users and/or on the context the virtual content elements.

According to at least one embodiment, the system may modify the mixed reality session with virtual content by injecting the selected content into the mixed reality environment. The system may modify the mixed reality session by interfacing directly with the mixed-reality program hosting and/or executing the mixed-reality session, through for example an application programming interface, such that the system may provide or identify virtual content elements to inject into the mixed reality experience to the mixed-reality program, and instruct the mixed-reality program to add the virtual content elements to the mixed reality session at a desired place and time. The system may continue to monitor the user's emotional state, and if the deviated emotions do not return within the expected baseline, the system may select and add additional virtual content elements until the user's emotional state once again falls within the expected baseline. The system may record the effect of the injected content element and provide the effect as feedback to the machine learning model. In some embodiments of the invention, the system may place the user into a mixed reality “lobby,” which may be a mixed reality experience created by the system and placed between or before a mixed-reality experience of the mixed reality session, which the system may modify to include virtual content elements to influence the user's emotional state prior to the user entering the next mixed reality experience.

In some embodiments of the invention, for example where multiple mixed-reality experiences such as training simulations, or movie vignettes, or game sections are designed to be experienced in succession, the system may order the successive content such that an emotional state of users after completing a first mixed-reality experience aligns with the expected baseline of a second mixed-reality experience. In other words, the system may purposefully pick scenarios based on a training plan to keep a user's behavior in a certain way. For example, if an intense training simulation often leaves users feeling anxious, and a high-intensity training simulation has an expected baseline of a state of anxiety, the system may order the training simulations such that the user experiences the intense training simulation before the high-intensity training simulation.

In some embodiments of the invention, an exemplary use case of the system may be illustrated in the following example:

Rob is a student at a first responder academy which has recently started using mixed reality scenarios to train the new recruits. Rob opts into a training module that is customized to ensure his mental state and resulting behavior matches an expected baseline mental state and behavior in a variety of simulated scenarios. For instance, one scenario may be responding to a car accident, while the second scenario is rescuing a cat from a tree. If Rob experiences the first scenario immediately before the second scenario, he may enter into the scenario of rescuing the cat from a tree with residual emotions such as anxiousness from the first scenario. The system attempts to bring Rob's baseline temperament from anxiousness to calmness by identifying soothing virtual content elements from Rob's profile, and projecting that soothing content into Rob's mixed reality session by displaying soothing images of natural landscapes and playing his preferred soothing music so that Rob is properly primed and calm for the next scenario, and can perform at his best.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation, or garbage collection, but this does not render the storage device as transitory because the data is not transitory while it is stored.

The following described exemplary embodiments provide a system, method, and program product to create an emotional profile tailored to a user, and inject emotionally-influencing situation-specific content into a mixed reality environment of the user based on the profile to maintain the user's emotional state at an expected baseline.

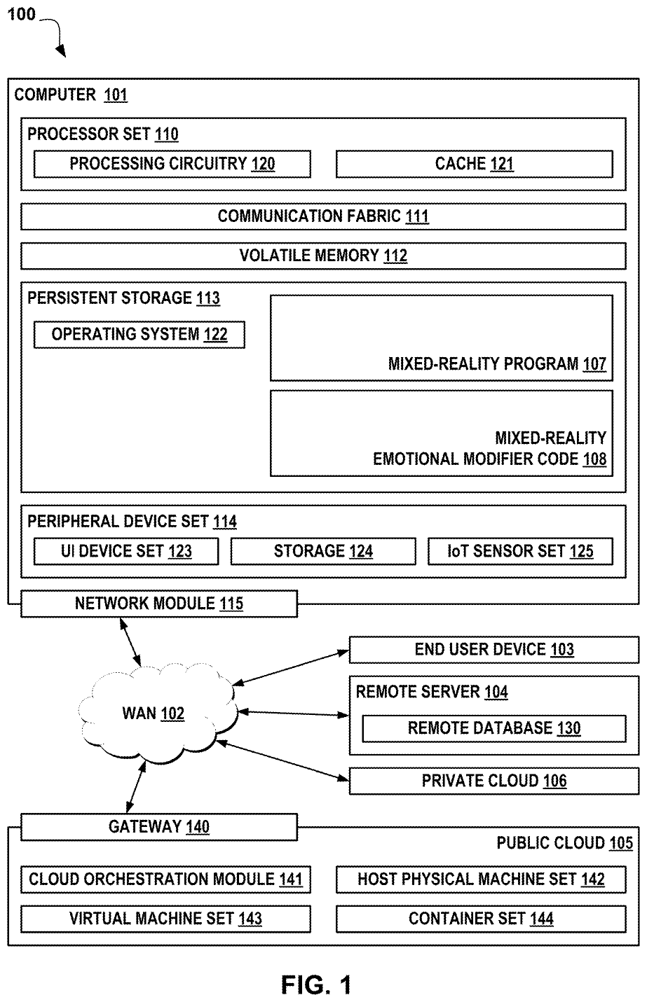

Referring now to FIG. 1, computing environment 100 contains an example of an environment for the execution of at least some of the computer code involved in performing the inventive methods, such as mixed-reality emotional modifier code 108. In addition to mixed-reality emotional modifier code 108, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and mixed-reality emotional modifier code 108, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer, or any other form of computer or mobile device now known or to be developed in the future that is capable of running a program, accessing a network, or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in mixed-reality emotional modifier code 108 in persistent storage 113.

COMMUNICATION FABRIC 111 is the signal conduction paths that allow the various components of computer 101 to communicate with each other. Typically, this fabric is made of switches and electrically conductive paths, such as the switches and electrically conductive paths that make up busses, bridges, physical input/output ports, and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data, and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in block mixed-reality emotional modifier code 108 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles, mixed reality headsets, and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications, including biometric sensors. For example, one sensor may be an infrared camera and another sensor may be a heart rate monitor.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

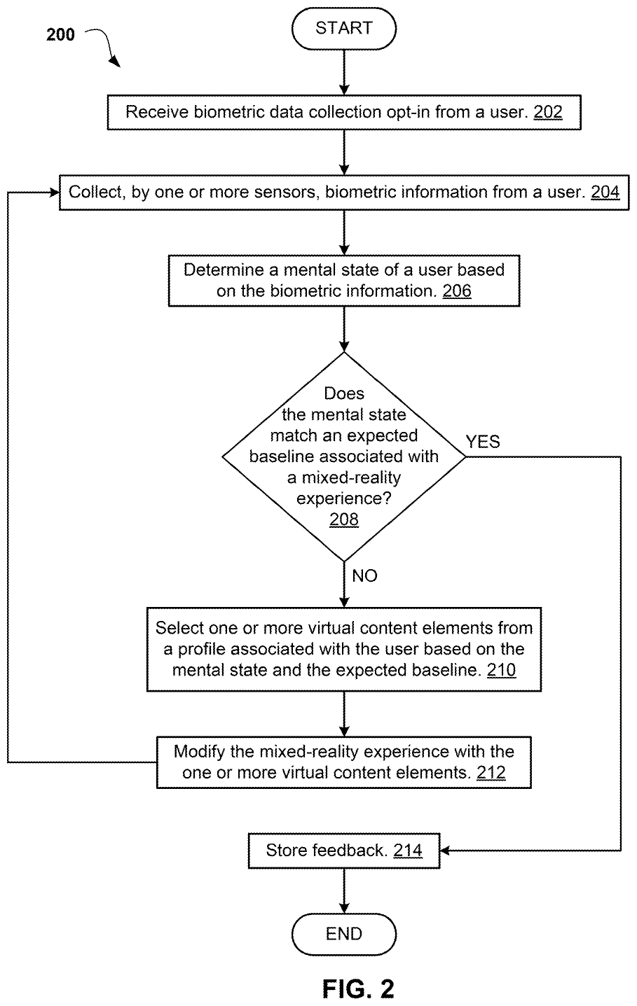

According to the present embodiment, the mixed-reality emotional modifier code 108 may be a program enabled to create an emotional profile tailored to a user, and inject emotionally-influencing situation-specific content into a mixed reality environment of the user based on the profile to maintain the user's emotional state at an expected baseline. The mixed-reality emotional modifier code 108 may, when executed, cause the computing environment 100 to carry out a mixed-reality emotional modifier process 200. The mixed-reality emotional modifier process 200 is explained in further detail below with respect to FIG. 2.

Referring now to FIG. 2, an operational flowchart illustrating a mixed-reality emotional modifier process 200 is depicted according to at least one embodiment. At 202, the mixed-reality emotional modifier code 108 receives biometric information collection opt-in from a user. Here, the mixed-reality emotional modifier code 108 may receive from a user explicit consent and permission for mixed-reality emotional modifier code 108 to collect biometric information on the user. The mixed-reality emotional modifier code 108 may prompt the user for such consent using a text and/or graphical prompt, for instance on the user's mobile device, which may detail the types, times, and contexts of biometric information collection that the mixed-reality emotional modifier code 108 would collect. In response to the prompt, the user may indicate either full or partial consent to biometric information collection. The mixed-reality emotional modifier code 108 may only collect biometric information that the user has consented to and may only use such collected biometric information for purposes communicated to the user.

At 204, the mixed-reality emotional modifier code 108 collects, by one or more sensors, biometric information from a user. The mixed-reality emotional modifier code 108 may collect biometric information through one or more sensors which may be standalone devices or may be integrated into devices such as wearable vitality trackers, mixed reality devices, mobile devices, et cetera. Biometric information of a user may be collected continuously or at regular intervals, for example during a mixed reality session. In some embodiments of the invention, biometric data may be collected at points indicated by an author of a mixed-reality experience where a user's emotional state is intended to be at an expected baseline, such as the story beats of a narrative experience.

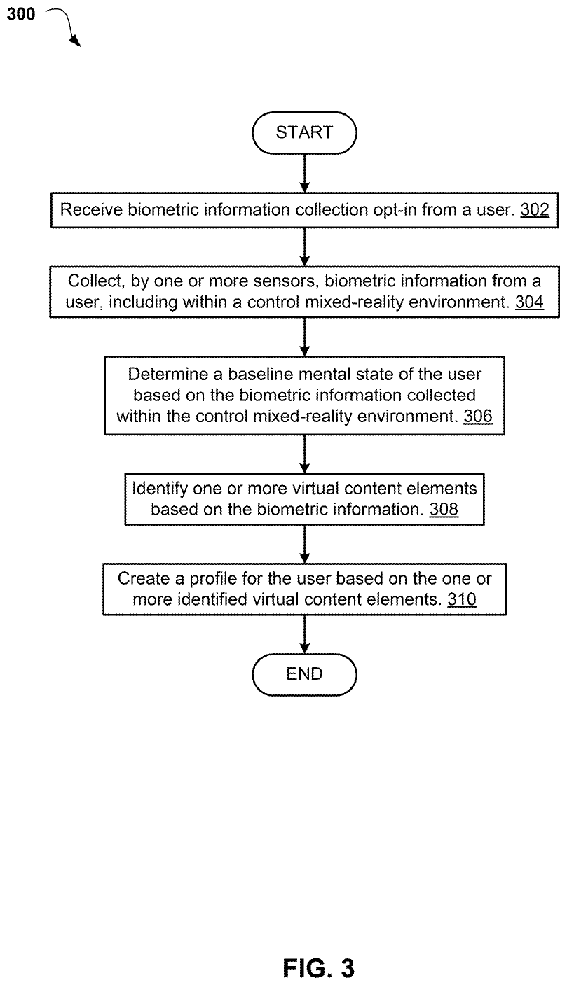

At 206, the mixed-reality emotional modifier code 108 determines a mental state of a user based on the biometric information. Here, mixed-reality emotional modifier code 108 may assess the emotional state of the user based on the biometric information by comparing the gathered biometric information against a default emotional state. The default emotional state may be discussed further with respect to FIG. 3 below. The mixed-reality emotional modifier code 108 may assess the emotional state upon initialization of a mixed-reality experience, in response to biometric information being received from sensors, at regular intervals, et cetera. The mixed-reality emotional modifier code 108 may initially use generic relationships between a generic user's biometric information and a generic user's mental state and may adjust the relationships over time based on the user's biometric information to better represent the connection between the biometric information and mental state of the individual user. The mixed-reality emotional modifier code 108 may use a machine learning model trained on generic relationships to identify the mental state of the user based on the user's biometric information.

At 208, the mixed-reality emotional modifier code 108 determines whether the mental state of the user matches an expected baseline associated with a mixed reality experience. the expected baseline may be an emotional state that is associated with a given mixed-reality program and/or mixed reality environment. The expected baseline may be an emotional state that an author of the mixed-reality program or mixed-reality environment expects an emotional state of a user to conform to immediately prior to initializing, for the duration of, or at one or more points within, the mixed reality experience. In some embodiments, the mixed-reality emotional modifier code 108 may not allow a user to begin a training scenario until the user's emotional state falls within the threshold values or otherwise conforms to the expected baseline.

The mixed-reality emotional modifier code 108 may determine whether the emotional state matches the expected baseline by comparing the emotions comprising the emotional state against the emotions comprising the expected baseline. The mixed-reality emotional modifier code 108 may determine that the emotional state matches the expected baseline if all of the emotions comprising the expected baseline are present in the emotional state, and/or when the magnitude of the emotions comprising the mental state are all within a threshold value of the magnitude of the emotions comprising the expected baseline. The threshold value may represent a difference between the magnitude of an emotion of the emotional state of the user and a magnitude of that emotion within the expected baseline that is large enough to conclude that the expected baseline and the emotional state do not match and that a deviation is present. The threshold value may be pre-provided, for example by the author, and/or may be determined by machine learning. The mixed-reality emotional modifier code 108 may compare each emotion of the expected baseline against the corresponding emotions of the user's emotional state and may identify that the measured emotional state has deviated from the expected baseline if any one emotion exceeds the threshold. In some embodiments of the invention, the mixed-reality emotional modifier code 108 may identify deviation if a plurality or a specified combination of emotions exceeds the threshold. In some embodiments of the invention, the mixed-reality emotional modifier code 108 may identify a deviation when one or more emotions comprising the expected baseline are not present in the emotional state.

According to one implementation, if the mixed-reality emotional modifier code 108 detects that the mental state of the user does not match an expected baseline associated with a mixed reality experience (step 208, “NO” branch), the mixed-reality emotional modifier code 108 may continue to step 210 to select one or more virtual content elements from a profile associated with the user based on the mental state and the expected baseline. If the mixed-reality emotional modifier code 108 determines that the mental state of the user does match an expected baseline associated with a mixed reality experience (step 208, “YES” branch), the mixed-reality emotional modifier code 108 may continue to step 214 to store feedback.

At 210, the mixed-reality emotional modifier code 108 selects one or more virtual content elements from a profile associated with the user based on the mental state and the expected baseline. The mixed-reality emotional modifier code 108 may identify which emotion or emotions deviated from the expected baseline, and the magnitude of the deviations, and assess what emotions and/or magnitude of effect must be produced on the user with respect to the deviating emotions to restore the emotional state to the expected baseline. The mixed-reality emotional modifier code 108 may consult the profile to determine which virtual content elements would produce the desired effect, based on their associated emotion, and/or the magnitude of effect the virtual content element would produce on the user or on generic users and/or on the context the virtual content elements. The profile may be discussed further with respect to FIG. 3 below.

At 212, the mixed-reality emotional modifier code 108 modifies the mixed reality experience with the one or more virtual content elements. The mixed-reality emotional modifier code 108 may modify the mixed reality session with virtual content by injecting the selected virtual content elements into the mixed reality environment. The mixed-reality emotional modifier code 108 may modify the mixed reality session by interfacing directly with the mixed-reality program hosting and/or executing the mixed-reality session, through for example an application programming interface, such that the mixed-reality emotional modifier code 108 may provide or identify virtual content elements to inject into the mixed reality experience to the mixed-reality program, and instruct the mixed-reality program to add the virtual content elements to the mixed reality session at a desired place and time.

At 214, the mixed-reality emotional modifier code 108 submits feedback. Here, the mixed-reality emotional modifier code 108 may, based on the biometric information, identify an effect that added virtual content elements produced on the user after being injected into the mixed-reality session, and may record whether the added virtual content elements were individually successful or unsuccessful at restoring the user's emotional state to the expected baseline. The mixed-reality emotional modifier code 108 may transmit the feedback to be stored in the profile and/or to train one or more machine learning models to improve the accuracy of predicting an effect that a virtual content element may have on the user.

Referring now to FIG. 3, an operational flowchart illustrating a profile creation process 300 is depicted according to at least one embodiment. At 302, the mixed-reality emotional modifier code 108 receives biometric information collection opt-in from a user. Here, the mixed-reality emotional modifier code 108 may receive from a user explicit consent and permission for mixed-reality emotional modifier code 108 to collect biometric information on the user. The mixed-reality emotional modifier code 108 may prompt the user for such consent using a text and/or graphical prompt, for instance on the user's mobile device, which may detail the types, times, and contexts of biometric information collection that the mixed-reality emotional modifier code 108 would collect. In response to the prompt, the user may indicate either full or partial consent to biometric information collection. The mixed-reality emotional modifier code 108 may only collect biometric information that the user has consented to, and may only use such collected biometric information for purposes communicated to the user.

At 304, the mixed-reality emotional modifier code 108 collects, by one or more sensors, biometric information from a user, including within a control mixed-reality environment. The mixed-reality emotional modifier code 108 may collect biometric information through one or more sensors which may be standalone devices or may be integrated into devices such as wearable vitality trackers, mixed reality devices, mobile devices, et cetera. Biometric information of a user may be collected continuously or at regular intervals, for example during a mixed reality session. In some embodiments of the invention, biometric data may be collected at points indicated by an author of a mixed-reality experience where a user's emotional state is intended to be at an expected baseline, such as the story beats of a narrative experience.

At 306, the mixed-reality emotional modifier code 108 determines a default mental state of the user based on the biometric information collected within the control mixed-reality environment. The control mixed-reality environment may be a mixed-reality environment that is designed not to provoke any emotional response from the user by default. The mixed-reality emotional modifier code 108 may monitor the user's biometric information while the user is within this environment, thereby assessing a default emotional state. The default emotional state may be a default emotional state associated with the user; the mixed-reality emotional modifier code 108 may identify the emotional state of the user by comparing biometric information against the default emotional state. In some embodiments of the invention, the mixed-reality emotional modifier code 108 may administer virtual content elements generically associated with certain emotions to the user in the control mixed-reality environment while measuring biometric information of the user, thereby preliminarily assessing which virtual content elements provoke which changes in the emotional state of the user, and the strength of such changes. In some embodiments of the invention, the default emotional state may be pre-determined and/or may be pre-provided by a programmer, the user, or an author of a mixed reality program or environment, and may be tailored to fit the user over time based on machine learning and/or biometric information.

At 308, the mixed-reality emotional modifier code 108 identifies one or more virtual content elements based on the biometric information. The mixed-reality emotional modifier code 108 may utilize the biometric information to identify changes in the user's emotional state that may be linked to particular virtual content elements. For example, if a user's emotional state changes after exposure to a particular music or color or image, the mixed-reality emotional modifier code 108 may identify that virtual content element and corresponding emotion to include in the profile. The mixed-reality emotional modifier code 108 may also identify one or more virtual content elements based on user preferences. The mixed-reality emotional modifier code 108 may obtain user preferences from the user or an online repository or web service. The user preferences may be interests, favorite music, favorite shows, fields of study, et cetera which may be connected to particular emotions. The mixed-reality emotional modifier code 108 may identify virtual content elements based on the user preferences. For example, the mixed-reality emotional modifier code 108 may select the favorite songs or images of the user to include in the profile as virtual content elements that could be injected into a mixed reality environment to calm the user.

The mixed-reality emotional modifier code 108 may also identify virtual content elements for inclusion in the profile from a list and/or repository based on the effect that a virtual content element has on a generic user as derived through statistical analysis, or the effect that a virtual content element has on a generic user with one or more matching interests or traits of the user; the mixed-reality emotional modifier code 108 may then, for example by introducing the content element to the user in the control mixed-reality environment or in other mixed-reality environments, assess the particular effect that the virtual content element produces on the mental state of the user. According to at least one embodiment, the mapping of the virtual content elements to a generic user's mental state may be based on public historical responses which could be cataloged based on a larger population. Within certain scenarios, the use of crowdsourcing emotional responses may be utilized for certain types of content requirements. Based on the expected response from a demographic of generic users, the mixed-reality emotional modifier code 108 may contextually utilize virtual content elements to evoke certain emotional responses from the user community. In some embodiments of the invention, the mixed-reality emotional modifier code 108 may utilize private interactions for statistical analysis of crowdsourced emotional responses, by scrubbing the data of any personal information pertinent to the individual from which the data originated and anonymizing the data for statistical purposes of tool usage and metrics. In some embodiments of the invention, the mixed-reality emotional modifier code 108 may analyze social media platforms for feedback and comments which could help indicate situations that cause a certain desired emotional response. In some embodiments of the invention, virtual content elements may be associated with particular mixed reality programs or experiences; the mixed-reality emotional modifier code 108 may select virtual content elements associated with a given mixed reality program or experience based on determining that the user owns, has access to, and/or is initializing an instance of that mixed reality program or experience. The mixed-reality emotional modifier code 108 may also select virtual content elements to add to the profile as they are added or become available to the mixed-reality emotional modifier code 108.

At 310, the mixed-reality emotional modifier code 108 creates a profile for the user based on the one or more identified virtual content elements. The profile may be a list of identified virtual content elements and the one or more emotions that each virtual content element evokes in the user and/or generic users, as well as, in some embodiments, the magnitude to which the virtual content element evokes that emotion. The magnitude may be expressed as a number, which represents the amount to which the user's emotional state is affected by the virtual content in one or more contexts. The profile may further comprise metadata associated with the virtual content elements providing identifying information about each virtual content element, such as for example the type of virtual content element (audio, video, gameplay, et cetera), the emotion or emotions evoked by the virtual content element in generic users and/or the user, the mixed reality programs or experiences associated with the virtual content element and/or within which the virtual content element could be injected, et cetera.

It may be appreciated that FIGS. 2-3 provides only illustrations of individual implementations and do not imply any limitations with regard to how different embodiments may be implemented. Many modifications to the depicted environments may be made based on design and implementation requirements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method for mixed-reality emotional modification, the method comprising:
collecting biometric information on a user;
identifying a mental state of the user based on the biometric information; and
responsive to determining that the mental state does not match an expected baseline associated with a mixed-reality experience, modifying the mixed-reality experience with one or more virtual content elements.

2. The method of claim 1, wherein the virtual content elements are selected from a profile based on a predicted emotional effect the virtual content element will have on the emotional state.

3. The method of claim 2, wherein the predicted emotional effect is based on one or more personal interests of the user.

4. The method of claim 1, wherein a plurality of expected baselines are associated with the mixed-reality experience.

5. The method of claim 1, wherein the identifying is based on comparing the biometric information against a default mental state.

6. The method of claim 1, wherein the determining occurs prior to instantiating the mixed-reality experience.

7. The method of claim 6, further comprising:
responsive to determining that the emotional state does match the expected baseline, disabling the user from accessing the mixed-reality experience.

8. A computer system for mixed-reality emotional modification, the computer system comprising:
one or more processors, one or more computer-readable memories, one or more mixed-reality devices, one or more sensors, one or more computer-readable tangible storage medium, and program instructions stored on at least one of the one or more tangible storage medium for execution by at least one of the one or more processors via at least one of the one or more memories, wherein the computer system is capable of performing a method comprising:
collecting biometric information on a user;
identifying a mental state of the user based on the biometric information; and
responsive to determining that the mental state does not match an expected baseline associated with a mixed-reality experience, modifying the mixed-reality experience with one or more virtual content elements.

9. The computer system of claim 8, wherein the virtual content elements are selected from a profile based on a predicted emotional effect the virtual content element will have on the emotional state.

10. The computer system of claim 9, wherein the predicted emotional effect is based on one or more personal interests of the user.

11. The computer system of claim 8, wherein a plurality of expected baselines are associated with the mixed-reality experience.

12. The computer system of claim 8, wherein the identifying is based on comparing the biometric information against a default mental state.

13. The computer system of claim 8, wherein the determining occurs prior to instantiating the mixed-reality experience.

14. The computer system of claim 13, further comprising:
responsive to determining that the emotional state does match the expected baseline, disabling the user from accessing the mixed-reality experience.

15. A computer program product for mixed-reality emotional modification, the computer program product comprising:
one or more computer-readable tangible storage media and program instructions stored on at least one of the one or more tangible storage media, the program instructions executable by a processor to cause the processor to perform a method comprising:
collecting biometric information on a user;
identifying a mental state of the user based on the biometric information; and
responsive to determining that the mental state does not match an expected baseline associated with a mixed-reality experience, modifying the mixed-reality experience with one or more virtual content elements.

16. The computer program product of claim 15, wherein the virtual content elements are selected from a profile based on a predicted emotional effect the virtual content element will have on the emotional state.

17. The computer program product of claim 16, wherein the predicted emotional effect is based on one or more personal interests of the user.

18. The computer program product of claim 15, wherein a plurality of expected baselines are associated with the mixed-reality experience.

19. The computer program product of claim 15, wherein the identifying is based on comparing the biometric information against a default mental state.

20. The computer program product of claim 15, wherein the determining occurs prior to instantiating the mixed-reality experience.
